(19) United States
(12) Patent Application Publication
(10) Pub. No.: US 2007/0043313 A1
(43) Pub. Date: Feb. 22, 2007

(54) Arm support apparatus

(76) Inventor: Guy Avon, Mascouche (CA)

Correspondence Address: Franz BONSANG;c/o PROTECTIONS EQUINOX INT'L INC., Suite 224, 4480, Cote-de-Liesse, Montreal, QC, H4N 2R1, CA

(21) Appl. No.: 11/586,553

(22) Filed: Oct. 26, 2006

Related U.S. Application Data
(63) Continuation-in-part of application No. 10/420,724, filed on Apr. 23, 2003.

(51) Int. Cl. A61F 5/00

(52) U.S. Cl. 602/20

(57) ABSTRACT

Arm support apparatus, for independently supporting an arm section and a forearm section of an injured arm of a user has a support body including elbow, forearm and wrist supports, a front portion located away from a torso of the user and a rear portion disposed towards the torso. The apparatus includes a first shoulder strap for supporting the forearm section and having strap ends connected respectively to a first front portion connector connected to the front portion and to a first rear portion connector connected to the rear portion, both located towards the wrist support. A second shoulder strap for supporting the arm section has strap ends connected respectively to a second front portion connector connected to the front portion towards the elbow support and spaced apart from the first front portion connector and to the body towards the wrist support.

### CROSS REFERENCE TO RELATED APPLICATIONS

This application is a continuation-in-part (C.I.P.) of co-pending patent application Ser. No. 10/420,724 filed on Apr. 23, 2003, now abandoned.

### FIELD OF THE INVENTION

The present invention concerns arm supports, more particularly to arm supports for hemiplegics with adjustable shoulder straps for independent support of the forearm and arm sections of an injured/paralyzed arm and a method for using same.

### BACKGROUND OF THE INVENTION

Hemiplegics suffer from paralysis on one side of the body. Typically, this type of paralysis results in loss of motor function and sensitivity in one arm or one leg or both limbs on the same side of the body. The extent of the loss of limb function depends upon the severity of the brain injury causing the paralysis. In the case of a paralyzed arm, orthopedic slings may be used to support the arm to prevent injury and to reduce movement relative to the body. Orthopedic slings for the arm typically have a support body to support the forearm, the elbow and the wrist and a number of shoulder straps connected to the support which loop either over one shoulder of the hemiplegic and/or around the neck. Many designs of this type of orthopedic sling exist, some of which are exemplified in the following:
U.S. Pat. No. 4,564,008, issued on Jan. 14, 1986 to Donahoo for “Arm Support Apparatus”;
U.S. Pat. No. 4,625,719, issued on Dec. 2, 1986 to Chambers for “Adjustable Arm Sling”;
U.S. Pat. No. 5,413,552, issued on May 9, 1995 to Iwuala for “Arm Sling with Humeral Stabilizer”; and
U.S. Pat. No. 5,569,172, issued Oct. 29, 1996 to Padden et al. for “Device for Supporting and Immobilizing a Patient's Arm and Shoulder and Method Thereof”.

The above designs, however, suffer from a number of significant drawbacks. Many of the strap systems used in the designs are not suitable for prolonged use since most have strap edges, which may either cut into an adjacent arm pit region or into an adjacent neck region, and which may cause fatigue and/or pain. Some strap systems are of a complex nature and require the use of a torso belt for further security. One problem with a torso belt appears to be bowing, such that when a user sits down, the strap may bow outwards making it easy for snagging and interference. The complex nature of some of the design's straps would significantly reduce the design's effectiveness and ease of use for the hemiplegic. Furthermore, some of the designs may only be limited to use in one position by having straps with limited adjustability. In addition, one design appears to have an opening against which the elbow would rest. Over prolonged periods of use, chaffing of the elbow region may result. Furthermore, most of the designs have straps that link the wrist region to the elbow region which allow the weight of the arm portion (including the shoulder) to shift downward while the wrist portion raises, and therefore induce the stretching of some shoulder ligaments (chronic risks of subluxation) that becomes painful to the hemiplegic over prolonged period of time, or increases the required time for recovery of the injured arm.

In general, the strap systems do not provide for sufficient protection against injuries on the arm and/or elbow occurring because of loss of sensitivity at these locations of the body.

Thus there is a need for an improved orthopedic arm support.

### SUMMARY OF THE INVENTION

The invention reduces the difficulties and disadvantages of the prior art by providing an arm support having shoulder straps, which are easy to assemble and which, when connected to the support, provide substantially increased comfort to the user. Two straps respectively connect to the elbow region and to the wrist region of the arm support in order to independently support the weights of the arm section and the forearm section of the injured or paralyzed arm. Furthermore, the support of the present invention is substantially rigid to avoid possible injuries to the arm and/or elbow of the user that would be caused by the loss of sensitivity. Advantageously, the previously described problems of straps cutting into the neck region and the upper arm, along with shoulder fatigue is substantially reduced or essentially eliminated. To accomplish this, a novel strap wrist connector arrangement is provided which allows the non-paralyzed arm to easily secure the support in place without the need for complex connecting methods. The healthy shoulder opposite the paralyzed arm is used to support the arm for extended periods of time without the need for extra belt straps. Moreover, another embodiment is provided in which the length of the support body can be customized so that the apparatus can to be custom-fit to many users. In addition, the apparatus is lightweight and inexpensive to manufacture using readily available materials.

In accordance with a first aspect of the present invention, there is provided an arm support apparatus for independently supporting an arm section and a forearm section of an injured arm of a user, said apparatus having a support body including an elbow support portion, a forearm support portion, a bottom portion and a wrist support portion, said support body having a front portion located away from a torso of the user and a rear portion disposed towards the torso, said apparatus comprising: first shoulder strap having a first strap first end portion and a first strap second end portion connecting respectively to said front portion towards said wrist support portion and to said rear portion towards said wrist support portion, and extending therebetween over a shoulder of the user, whereby the first shoulder strap generally supports the forearm section of the injured arm; and second shoulder strap having second strap first and second end portions, said second strap first end portion connecting to said front portion towards said elbow support portion, said second strap second end portion connecting to said support body adjacent said elbow support portion, whereby the second shoulder strap generally supports the arm section of the injured arm independently of the forearm section supported by the first shoulder strap, said second shoulder strap having sufficient length to pass over the shoulder of the user.

Conveniently, the shoulder is located opposite the injured arm of the user, said first and second shoulder straps having respectively sufficient length to pass over the opposite shoulder situated proximal said wrist support portion and distal said elbow support portion.

In one embodiment, the apparatus further includes: first front portion connector member connected to said front portion of said support body and first rear portion connector member connected to said rear portion of said support body, said first front portion and said first rear portion connector members being located towards said wrist support portion; second front portion connector member connected to said front portion of said support body and spaced apart from said first front portion connector member, said second front portion connector member being located towards said elbow support portion; an elbow connector loop connected to said elbow support portion; said first strap first and second end portions connecting to said first front and rear portion connector members, respectively; said second strap first end portion connecting to said second front portion connector member and said second strap second end portion extending through said elbow connector loop and connecting to said support body.

Typically, the apparatus further includes a wrist end connector member connected to said support body and located towards said wrist support portion, said second strap second end portion extending through said elbow connector loop and connecting to said wrist end connector member.

In one embodiment, said first front portion connector member includes a first strap guide located in a front upper edge portion of said support body.

Typically, the apparatus further comprises a thumb strap having one thumb strap end secured to one side of said wrist support portion, the other thumb strap end being releasably connected to the other side of said wrist support portion.

In one embodiment, said wrist support portion is releasably connected to said forearm support portion.

In one embodiment, the apparatus further includes a shoulder pad connected to said first and second shoulder straps, said shoulder pad having a upper arcuate piece including respective guide slots through which said first and second shoulder straps pass.

Typically, the apparatus further includes an elbow cushion connected to said elbow support portion, and a forearm support cushion connected to said forearm support portion.

In one embodiment, said front portion includes a plurality of ventilation holes therein.

In one embodiment, the lengths of said first and second shoulder straps are adjustable.

Typically, said support body is generally rigid.

### BRIEF DESCRIPTION OF THE FIGURES

Further aspects and advantages of the present invention will become better understood with reference to the description in association with the following

Figures, wherein:

FIG. 1 is a front perspective view of a first embodiment of an arm support in use with a hemiplegic person;

FIG. 2 is a rear perspective view of FIG. 1;

FIG. 3 is a simplified front perspective view of the arm support;

FIG. 4 is a simplified, exploded view showing the rear of the arm support;

FIG. 5 is a simplified exploded view of a shoulder strap;

FIG. 5a is a section view taken along line 5a-5a of FIG. 5;

FIG. 6 is a front perspective view of a second embodiment of an arm support;

FIG. 7 is a simplified view showing the front of the arm support of FIG. 6;

FIG. 8 is a simplified, exploded perspective rear view of the arm support of FIG. 6;

FIG. 9 is a simplified perspective front view of the arm support of FIG. 6 showing an elbow support and a wrist support;

FIG. 10 is a simplified perspective rear view showing an elbow support and a wrist support; and

FIG. 11 is a simplified exploded perspective view of the arm support with a belt and belt connectors.

### DETAILED DESCRIPTION OF THE PREFERRED EMBODIMENTS

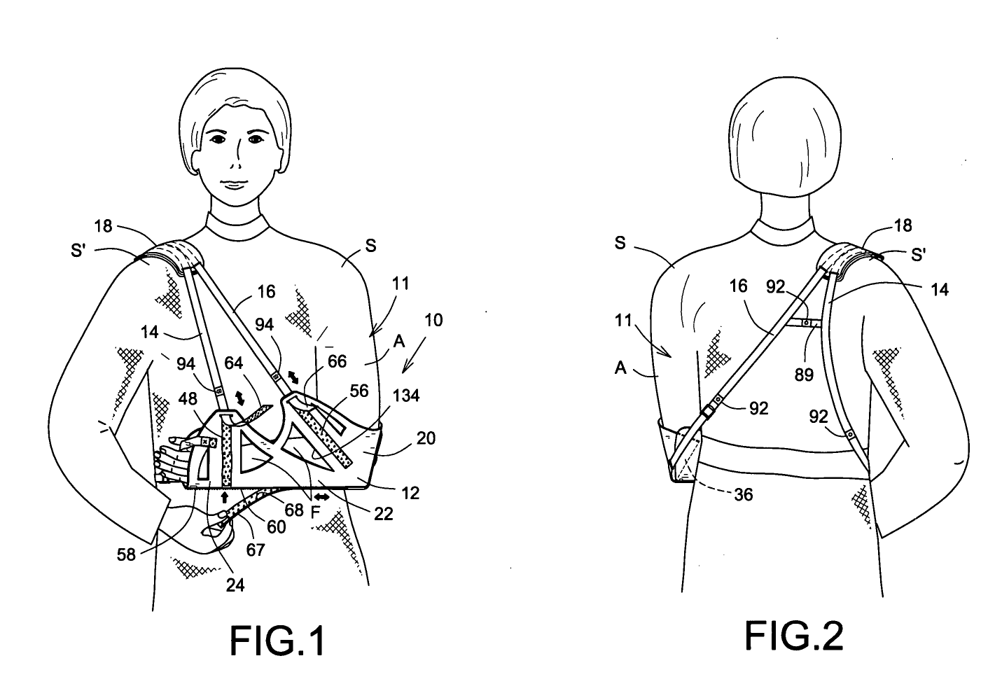

Referring to FIG. 1, a first embodiment of an arm support apparatus is shown generally at 10. Broadly speaking, the apparatus 10 includes a support body 12, two distinct shoulder straps 14, 16 and a shoulder pad 18.

Referring now to FIGS. 1, 3, and 4, the support body 12 includes an elbow support portion 20, a forearm support portion 22 and a wrist support portion 24. Typically, the support body 12 is made from a resilient sheet of generally rigid material, such as any thermoplastics, which is shaped to produce a curved structure. The curved structure defines a lower inner sidewall 26, an elbow inner sidewall 28 and generally opposed front and rear inner sidewalls 30, 31, all of which are interconnected. The support body 12 has a front outer surface 32, which is disposed away from a torso of a user and a rear outer surface 34, which is disposed towards the torso. Although not specifically shown, the upper edge of the rear inner sidewall 31 is generally slightly bent inwardly to improve comfort to the user, especially when in the sit down position. The user's arm 11, when in place, rests against the lower inner sidewall 26, the two front and rear inner sidewalls 30, 31 and the elbow inner sidewall 28. For ease of disassembly for cleaning, a flap 36 extends around the elbow support portion 20. Typically, the flap 36 is releasably connected to the inside of the rear inner sidewall 31 using a quick release strip 38 such as Velcro™ or some other material known to those skilled in the art. The releasable connection allows for custom adjustments to the user, depending on the type of clothing being worn.

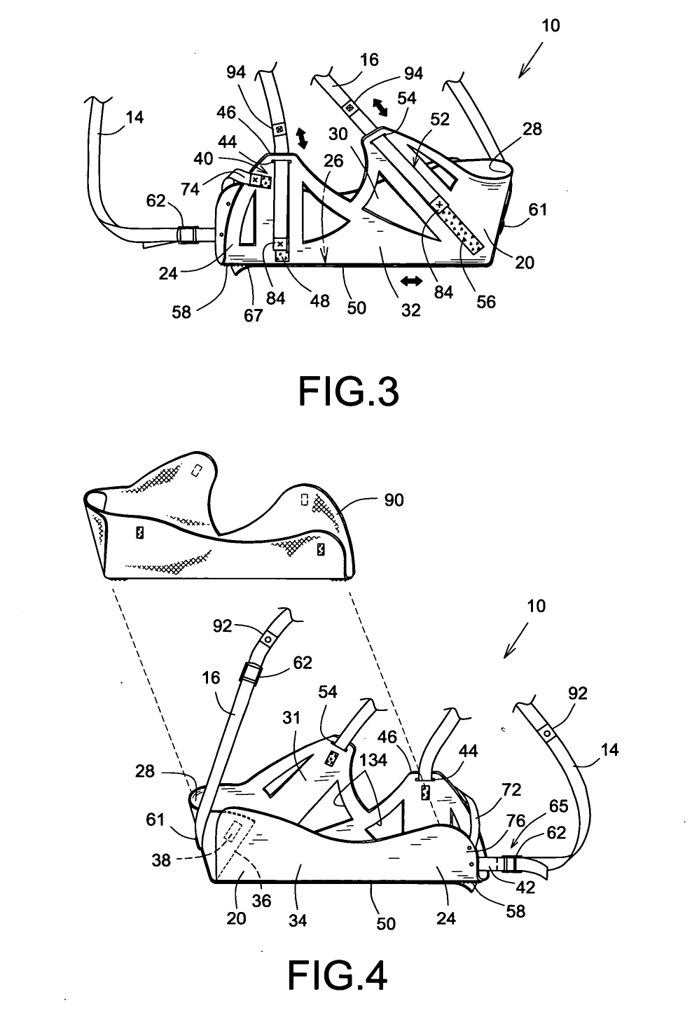

Referring to FIGS. 3 and 4, a first front portion connector member 40 is connected to the front outer surface 32 of the support body 12, near the wrist support portion 24, and a first rear portion connector member 42 is connected to one of the sidewalls 30 at the rear of the wrist support portion 24 of the support body 12. The first front portion connector member 40 includes a first strap guide, preferably a slot 44, cut into an upper edge portion 46 at the front of the support body 12, and a first front connector strip 48 extending from the first slot 44 towards a bottom portion 50 of the support body 12. The first front connector strip 48 is attached to the front outer surface 32 of the support body 12. A second front portion connector member 52 is connected to the front outer surface 32 of the support body 12. The second member 52 is spaced apart from the first front portion connector 40, and is near the elbow support portion 20. The second front portion connector member 52 includes a second strap guide, preferably a slot 54 located in the front upper edge portion 46 of the support body 12 and away from the first slot 44. The second front portion connector member 52 also includes a second front connector strip 56 extending from the second slot 54 towards the bottom outer surface of the support body 12. The second front connector strip 56 is angled away from the first front connector strip 48 towards the elbow support portion 20. Obviously, any other type of first and second strap guides 44, 54, such as connector loops or the like, well known to one skilled in the art could be used without departing from the scope of the present invention.

A wrist end connector member 58 is connected to the support body 12 and located on the bottom portion 50 towards the wrist support portion 24.

The bottom portion 50 of the support body 12 includes a bottom connector strip 60, as best seen in FIG. 1, which extends substantially from the elbow support portion 20 towards the wrist end connector member 58. A connector loop 61 is connected to the elbow support portion 20 for connecting and securing the second shoulder strap 16 to the outer surface of the elbow support portion 20.

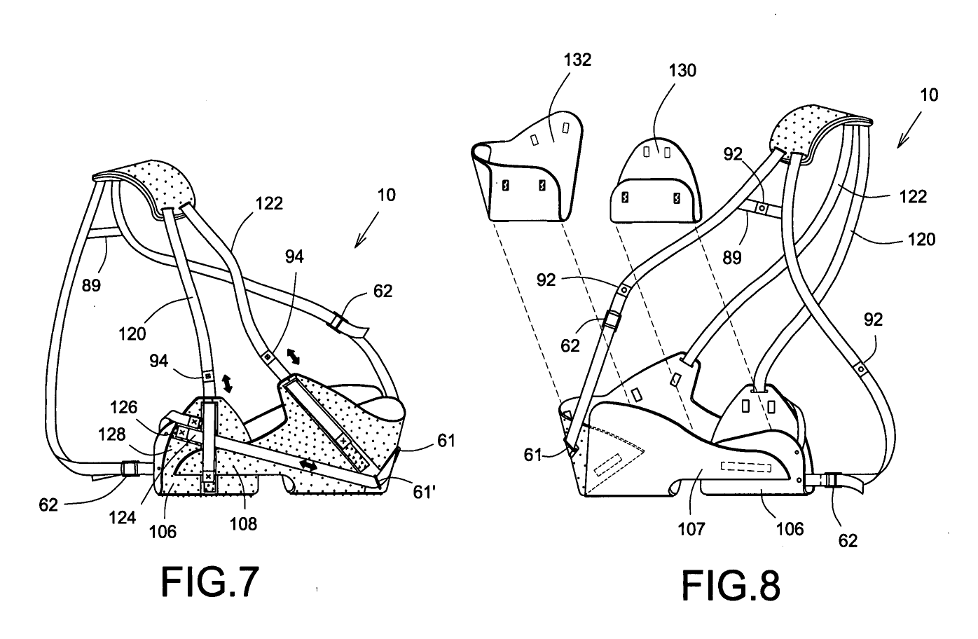

As best illustrated in FIGS. 1, 2, 3 and 4, the first and second shoulder straps 14, 16 are sufficiently long to pass over one shoulder of a user, preferably the healthy shoulder S′ opposite from the injured arm 11, and further behind the user's torso for the second shoulder strap 16. The straps each 14, 16 have a length adjuster 62 located in the strap. The first shoulder strap 14 has a first strap first end portion 64, which includes a Velcro™ strip for connecting to the connecting strip 48 of first front portion connecting member 40 and a first strap second end portion 65, which is connected to first rear portion connector member 42. Specifically, to support the forearm section F of the injured arm 11, first shoulder strap 14 extends between connecting members 40, 42 over a proximal shoulder S′, opposite the supported injured or paralyzed arm 11, of a user on a single proximal side of the torso of said user, the proximal side and the proximal shoulder S′ being situated proximal wrist support portion 24 and distal said elbow support portion 20. The second shoulder strap 16 has second strap first and second end portions 66 and 67. The second strap first end portion 66 is connected to the second front portion connector member 52, and includes a Velcro™ strip for connecting to the second front connector strip 56. The other second strap end portion 67, i.e. second strap second end portion, is connected to the elbow support portion 20 (via the connector loop 61) with attachment to the wrist end connector member 58. The second strap second end portion 67 includes a first connector strip 68, which is releasably connected to the bottom connector strip 60 along substantially the entire length of the bottom connector strip 60. As shown more specifically in FIGS. 1, 2, and 6-8, the first and second straps 14, 16 independently support the weights of the forearm section F and the arm section A (including shoulder S corresponding thereto) of the injured arm 11, respectively.

Referring to FIGS. 3 and 4, a thumb strap 72 is connectable over the thumb of the user when the arm 11 is in place for increased comfort and stability of the arm. The thumb strap 72 has one end 73, which is fixed to the one side of the wrist support portion 24, the other end is releasably connected to a thumb strap connector strip 74.

For ease of release, each of the strap ends may include a tab 84 located at the strap end, but which is not attachable to the connecting strips. The tabs 84 allow the user to pull up against the tab to remove the strap from the connecting strip and to pull the straps down over the connecting strips. The connector strips used throughout are typically made of Velcro™, although one skilled in the art will recognize that many types of quick release mechanisms may be used without deviating from the scope of the invention.

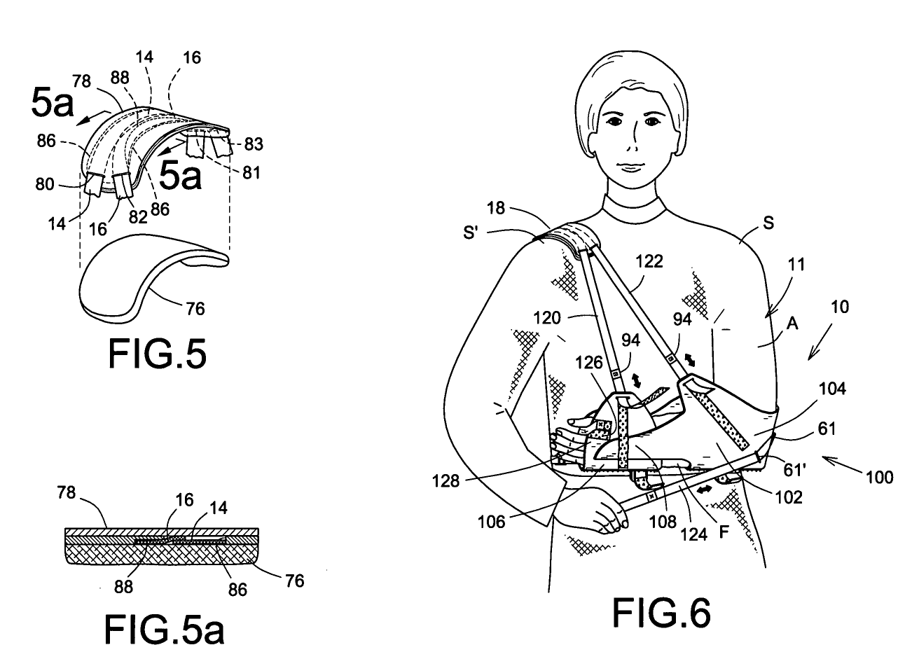

Referring now to FIGS. 1 and 5, the shoulder pad 18 is located on one shoulder S′ opposite a supported arm 11 and is connected to the first and second shoulder straps 14, 16. The shoulder pad 18 includes an arcuate cushion 76, which is connected to an upper arcuate piece 78, which is typically a piece of plastic material. The arcuate piece 78 includes four respective guide slots 80, 81, 82, and 83 located at opposite ends of the arcuate piece 78. Guide slots 82, 83 are generally inclined relative to the guide slots 80, 81. The first shoulder strap 14 engages the guide slots 80 and 81, while the second shoulder strap 16 passes through and is directionally guided by the generally inclined guide slots 82 and 83. The first shoulder strap 14 is connected substantially longitudinally between the guide slots 80 and 81 within an upper recess 86 and with a slight curve toward the second shoulder strap 16, whereas the second shoulder strap 16 is connected so that it generally curves towards and slightly over the first shoulder strap 14 between the inclined guide slots 82 and 83 inside the recess 86 because of the internal abutment shoulder 88, as shown in FIGS. 5 and 5a. The first shoulder strap 14 is slightly curved to retain the shoulder pad 18 away from the user's neck.

As shown in FIG. 2, a cross strap 89 connects the first and second shoulder straps 14, 16 and lies adjacent the upper back of the user. The cross strap 89, the shoulder straps 14, 16 and the shoulder pad 18 forms a generally A-shaped web. The cross-strap 89 ensures that the first shoulder strap 14 clears the back region of the user's armpit and that the second shoulder strap 16 does not pull the shoulder pad 18 against the user's neck.

Referring to FIG. 4, a one-piece cushion 90 may be connected to the Inside of the support body 12. The cushion 90 may also be separate depending upon the requirements of the user.

Operation

The device 10 is typically supplied to the user as shown in FIG. 1. The operation of the device 10 will now be described with reference to FIGS. 1 and 3.

The user passes the shoulder straps 14, 16 around the neck and positions the shoulder pad 18 against the shoulder S′ opposite the paralyzed arm 11. The non-paralyzed arm lifts the paralyzed arm and maneuvers it into the support body 12 so that the elbow and forearm lay snuggly against wrist support portion 24, the elbow support portion 20 and the forearm support portion 22. The first strap 14 is pulled through the first slot 44 and the Velcro™ strip is engaged with the Velcro™ strip on the front surface. Similarly, the second strap 16 is pulled down through the second slot 54 and under the support body 12. When the desired height and comfort level is reached, by finely adjusting the straps, the second strap 16 is connected to the wrist end connector 58, while supporting the elbow support portion via the connector loop 61. In order to ease this operation, the apparatus 10 is typically provided with color coded tabs or the like attached to the different straps. More specifically, the portions of the straps 14, 16, 89 that need to be located at the back of the user bear a first color coded tab 92, such as “red” for example, as shown in FIGS. 2 and 8. Similarly, the portions of the straps 14, 16 that need to be located in front of the user bear a second color-coded tab 94, such as “green” for example, as shown in FIGS. 1, 3 and 7. To release the arm from the support apparatus, the above-described process is reversed.

Alternatives

There may be applications in which length of the support body 12 needs to be universal, such as for custom fitting to different users. Accordingly, if the apparatus 10 is to be useful for more than one hemiplegic, the support body 12 length may also need to be extended or contracted. In these applications, the first embodiment 10 would be unsuitable. An alternative embodiment of an arm support apparatus of the present invention is shown generally at 100. The operation of the support apparatus 100 is essentially the same as with the first embodiment 10.

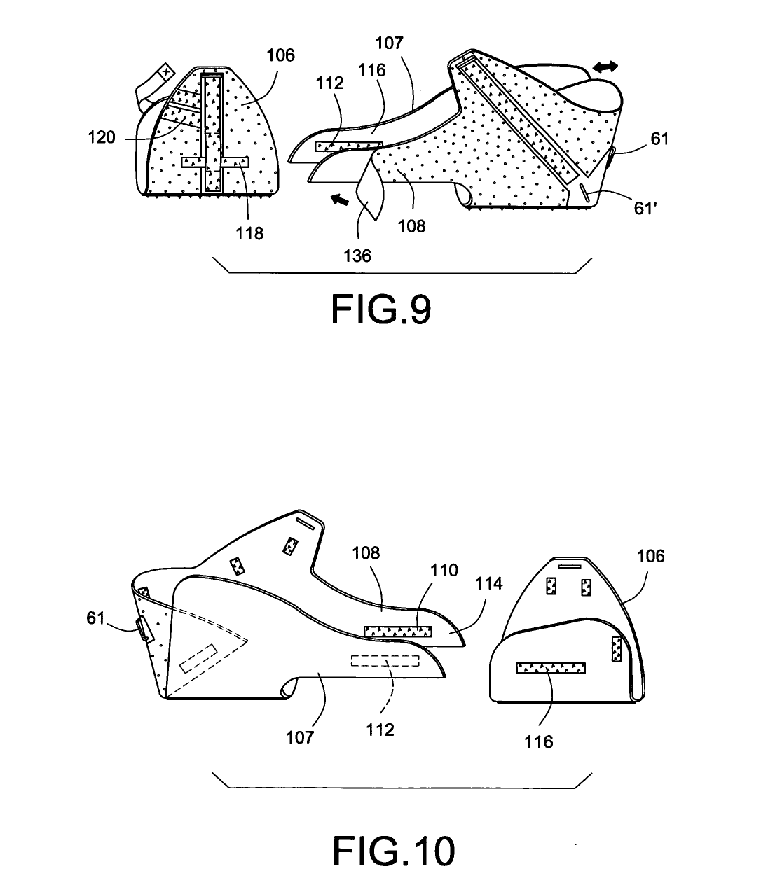

The salient structural differences between the two embodiments will now be described with reference to FIGS. 6 to 10. A support body 102 includes an elbow support portion 104, which is separable from a wrist support portion 106. The elbow support portion 104 includes first and second connector arms 107, 108, which extend away from the elbow support portion 104. The first and second connector arms 107, 108 are spaced apart to receive therebetween the wrist support portion 106. The first and second connector arms 107, 108 each have first and second wrist support portion connectors 110, 112 located on respective arm inner surfaces 114, 116. The wrist support portion 106 includes first and second forearm connectors 116, 118 located on the front and rear portions to connect with the first and second wrist support connectors 110, 112.

In this embodiment, a first strap 120 connects to the support body as described for the first embodiment 10. A second strap 122 includes a second strap end 124, which is connectable to a wrist end connector member 126 located on a wrist support outer surface 128. When connected to the wrist end connector member 126, the second strap 122 lies across the front of the support body, passing through connector loops 61, 61′. As with the first embodiment 10, a forearm cushion 130 and an elbow cushion 132 may be releasably connected to the inside of the support body.

As best illustrated in FIG. 1, for greater comfort and improved aesthetic appearance, the front portion 22 of the support body may include a plurality of ventilation holes 134 therein. A patterned cover 136 can be releasably adhered to the outer surfaces, as well as to the shoulder pad 18, to improve the aesthetic appearance of the apparatus and make the latter more “invisible” to others. Different patterned covers 136 can be used in different occasions to fit the clothing of the user. The apparatus is typically made from lightweight, malleably, easy-to-clean materials such as thermoplastics and the like.

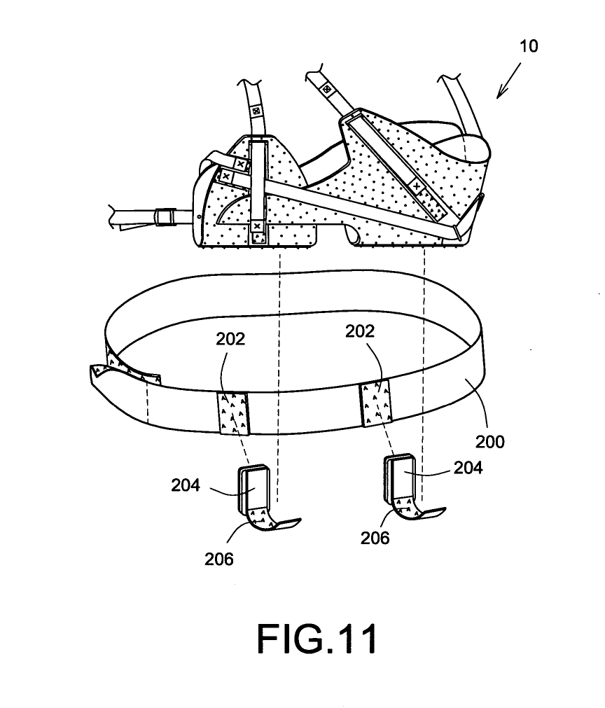

Referring now to FIG. 11, two releasable belt connectors 204 may be releasably connected to the bottom connector strip 60. The belt connectors 204 have Velcro™ strips 206 for connecting to the connector strip 60. This enables the user to further secure the support body against the body using a belt 20, which also has Velcro™ strips 202 located thereon. Also, the belt connectors 204 may provide temporary support to the support body should one of the straps require temporary removal or temporarily relief the user's shoulder S′ from supporting the weight of the paralyzed or injured arm 11 and transfer its weight on the belt 20. Obviously, the belt connectors 204 could be configured and sized to simply support the apparatus 10 without the use of strips 206.

I claim:

1. An arm support apparatus for independently supporting an arm section and a forearm section of an injured arm of a user, said apparatus having a support body including an elbow support portion, a forearm support portion, a bottom portion and a wrist support portion, said support body having a front portion located away from a torso of the user and a rear portion disposed towards the torso, said apparatus comprising:
first shoulder strap having a first strap first end portion and a first strap second end portion connecting respectively to said front portion towards said wrist support portion and to said rear portion towards said wrist support portion, and extending therebetween over a shoulder of the user, whereby the first shoulder strap generally supports the forearm section of the injured arm; and
second shoulder strap having second strap first and second end portions, said second strap first end portion connecting to said front portion towards said elbow support portion, said second strap second end portion connecting to said support body adjacent said elbow support portion, whereby the second shoulder strap generally supports the arm section of the injured arm independently of the forearm section supported by the first shoulder strap, said second shoulder strap having sufficient length to pass over the shoulder of the user.

2. The apparatus, according to claim 1, in which the shoulder is located opposite the injured arm of the user, said first and second shoulder straps having respectively sufficient length to pass over the opposite shoulder situated proximal said wrist support portion and distal said elbow support portion.

3. The apparatus, according to claim 2, further including:
first front portion connector member connected to said front portion of said support body and first rear portion connector member connected to said rear portion of said support body, said first front portion and said first rear portion connector members being located towards said wrist support portion;
second front portion connector member connected to said front portion of said support body and spaced apart from said first front portion connector member, said second front portion connector member being located towards said elbow support portion;
an elbow connector loop connected to said elbow support portion;
said first strap first and second end portions connecting to said first front and rear portion connector members, respectively;
said second strap first end portion connecting to said second front portion connector member and said second strap second end portion extending through said elbow connector loop and connecting to said support body.

4. The apparatus, according to claim 3, further including a wrist end connector member connected to said support body and located towards said wrist support portion, said second strap second end portion extending through said elbow connector loop and connecting to said wrist end connector member.

5. The apparatus, according to claim 4, in which said first front portion connector member includes a first strap guide located in a front upper edge portion of said support body.

6. The apparatus, according to claim 5, in which said first front portion connector member further includes a first front connector strip extending from said first strap guide towards said bottom portion of said support body.

7. The apparatus, according to claim 6, in which said first front connector strip is attached to said front portion of said support body.

8. The apparatus, according to claim 5, in which said second front portion connector member includes a second strap guide located in said front upper edge portion of said support body and away from said first strap guide.

9. The apparatus, according to claim 8, in which said second front portion connector member further includes a second front connector strip extending from said second strap guide towards said bottom portion of said support body.

10. The apparatus, according to claim 9, in which said second front connector strip is angled away from said first front connector strip towards said elbow support portion.

11. The apparatus, according to claim 10, in which said bottom portion of said support body includes a bottom connector strip extending substantially from said elbow support portion towards said wrist end connector member.

12. The apparatus, according to claim 11, in which said second strap second end end includes a first connector strip which is releasably connected to said bottom connector strip along substantially the entire length of said bottom connector strip.

13. The apparatus, according to claim 12, in which said second strap first end portion includes a second connector strip which is releasably connected to said second front connector strip.

14. The apparatus, according to claim 11, in which said wrist end connector member is connected to said front portion adjacent said first strap guide.

15. The apparatus, according to claim 11, in which said elbow support portion includes a flap releasably connected to said rear portion adjacent said elbow support portion.

16. The apparatus, according to claim 1, further comprising a thumb strap having one thumb strap end secured to one side of said wrist support portion, the other thumb strap end being releasably connected to the other side of said wrist support portion.

17. The apparatus, according to claim 16, in which said front portion of said support body adjacent said wrist support portion includes a thumb strap connector strip.

18. The apparatus, according to claim 1, in which said wrist support portion is releasably connected to said forearm support portion.

19. The apparatus, according to claim 18, in which said forearm support portion includes first and second connector arms extending away from said elbow support portion, said first and second connector arms being spaced apart to receive therebetween said wrist support portion, said first and second connector arms having first and second wrist support portion connectors.

20. The apparatus, according to claim 19, in which said wrist support portion include first and second forearms connectors located on said front and rear portions to connect with said first and second wrist support connectors.

21. The apparatus, according to claim 1, further including a shoulder pad connected to said first and second shoulder straps, said shoulder pad having a upper arcuate piece including respective guide slots through which said first and second shoulder straps pass.

22. The apparatus, according to claim 21, in which said first shoulder strap is connected generally longitudinally between opposite guide slots of said shoulder pad, said second shoulder strap being curved towards said first shoulder strap.

23. The apparatus, according to claim 22, in which a cross strap connects said first and second shoulder straps adjacent the upper back of the user, said cross strap, said shoulder straps and said shoulder pad forming a generally A-shaped web.

24. The apparatus, according to claim 23, in which the shoulder pad is located on one shoulder opposite the supported injured arm.

25. The apparatus, according to claim 1, further including an elbow cushion and a forearm cushion connected to said elbow support portion and said forearm support portion, respectively.

26. The apparatus, according to claim 12, in which first and second releasable belt connectors are releasably connected to said bottom connector strip and to a belt around the user's torso.

27. The apparatus, according to claim 1, in which said front portion includes a plurality of ventilation holes therein.

28. The apparatus, according to claim 1, in which the lengths of said first and second shoulder straps are adjustable.

29. The apparatus, according to claim 18, in which said wrist support portion and said forearm support portion of said support body are generally rigid.

30. The apparatus, according to claim 1, in which said support body is generally rigid.
